(19) United States
(12) Patent Application Publication
(10) Pub. No.: US 2012/0010539 A1
(43) Pub. Date: Jan. 12, 2012

(54) OPERATION DEVICE AND SURGICAL APPARATUS

(76) Inventors: Chie YACHI, Fuchu-shi (JP); Yoshitaka FUJII, Atsugi-shi (JP)

(21) Appl. No.: 13/236,705

(22) Filed: Sep. 20, 2011

Related U.S. Application Data
(63) Continuation of application No. PCT/JP2010/056335, filed on Apr. 7, 2010.

(51) Int. Cl. A61N 7/00; A61B 17/00

(52) U.S. Cl. 601/2; 606/1

(57) ABSTRACT

An operation device configured to control driving of an apparatus, includes a switch contact point which is provided in a fixed handle, and a switch operation portion which includes a pressure-receiving portion configured to operate the switch contact point between an open state and a closed state in accordance with a pressing state of the pressure-receiving portion. The operation device includes a support portion configured to support the pressure-receiving portion in such a manner that the pressure-receiving portion can incline and move with respect to the fixed handle about an inclination movement center, the support portion being configured to support the pressure-receiving portion in such a manner that the user can press the pressure-receiving portion in a direction where the inclination movement center of the pressure-receiving portion is arranged in a state in which the pressure-receiving portion is inclined and moved and in a neutral position state.

### CROSS-REFERENCE TO RELATED APPLICATIONS

This application is a Continuation Application of PCT Application No. PCT/JP2010/056335, filed Apr. 7, 2010 and based upon and claiming the benefit of priority from prior U.S. patent application Ser. No. 12/427,824, filed Apr. 22, 2009, the entire contents of all of which are incorporated herein by reference.

### BACKGROUND OF THE INVENTION

1. Field of the Invention

The present invention relates to an operation device configured to control driving of an apparatus such as a surgical apparatus and a surgical apparatus including an operation portion.

2. Description of the Related Art

In general, there is a device as a surgical apparatus disclosed in, e.g., Jpn. Pat. Appln. KOKAI Publication No. 2002-291745. A main body operation portion of this device is provided with a switch configured to control driving of the device. The switch includes a pressure-receiving portion configured to be pressed by a user, an activation rod configured to be activated when the pressure-receiving portion is pressed, and a switch contact point. The pressure-receiving portion is provided at one end side of the activation rod, and the switch contact point is provided at the other end side of the activation rod. During operation in which a user pushes the pressure-receiving portion, the activation rod moves in an axial direction and presses the switch contact point. Thereby, the driving of the device is controlled.

### BRIEF SUMMARY OF THE INVENTION

According to one aspect of the invention, an operation device configured to control driving of an apparatus, includes that an operation portion housing which includes a fixed handle, and a movable handle which is pivotably supported with respect to the fixed handle and which is configured to be opened and closed with respect to the fixed handle, the operation portion housing being configured to be held by a user; a switch contact point which is provided in the fixed handle; a switch operation portion which includes a pressure-receiving portion which is provided on a surface of a distal end side of the fixed handle and which includes a pressing surface substantially formed in an arc shape and extended from a plane, which includes a central position of the movable handle in widthwise directions of the operation portion housing perpendicular to opening/closing directions of the movable handle, to a first side and a second side opposite to the first side, the switch operation portion being configured to operate the switch contact point between an open state and a closed state in accordance with a pressing state of the pressure-receiving portion; a support portion configured to support the pressure-receiving portion in such a manner that the pressure-receiving portion can incline and move, with respect to the fixed handle about an inclination movement center in the fixed handle, in two directions including a first inclination movement direction that is substantially perpendicular to the opening/closing directions of the movable handle and is corresponding to a tangential line direction of the pressing surface of the pressure-receiving portion and a second inclination movement direction opposite to the first inclination movement direction, the support portion being configured to support the pressure-receiving portion in such a manner that the user can press the pressure-receiving portion in a direction where the inclination movement center of the pressure-receiving portion is arranged in a state in which the pressure-receiving portion is inclined and moved and in a neutral position state in which the pressure-receiving portion is not inclined and moved; and a restricting section configured to restrict the inclination movement directions of the pressure-receiving portion so that the pressure-receiving portion can move in two directions including the first inclination movement direction and the second inclination movement direction.

According to one another aspect of the invention, a surgical apparatus includes that a hand piece which includes an operation portion configured to control driving, wherein the operation portion includes: an operation portion housing which includes a fixed handle, and a movable handle which is pivotably supported with respect to the fixed handle and which is configured to be opened and closed with respect to the fixed handle, the operation portion housing being configured to be held by a user; a switch contact point which is provided in the fixed handle; a switch operation portion which includes a pressure-receiving portion which is provided on a surface of a distal end side of the fixed handle and which includes a pressing surface substantially formed in an arc shape and extended from a plane, which includes a central position of the movable handle in widthwise directions of the operation portion housing perpendicular to opening/closing directions of the movable handle, to a first side and a second side opposite to the first side, the switch operation portion being configured to operate the switch contact point between an open state and a closed state in accordance with a pressing state of the pressure-receiving portion; a support portion configured to support the pressure-receiving portion in such a manner that the pressure-receiving portion can incline and move, about an inclination movement center in the fixed handle with respect to the fixed handle, in two directions including a first inclination movement direction that is substantially perpendicular to the opening/closing directions of the movable handle and is corresponding to a tangential line direction of the pressing surface of the pressure-receiving portion and a second inclination movement direction opposite to the first inclination movement direction, the support portion being configured to support the pressure-receiving portion in such a manner that the user can press the pressure-receiving portion in a direction where the inclination movement center of the pressure-receiving portion is arranged in a state in which the pressure-receiving portion is inclined and moved and in a neutral position state in which the pressure-receiving portion is not inclined and moved; and a restricting section configured to restrict the inclination movement directions of the pressure-receiving portion so that the pressure-receiving portion can move in two directions including the first inclination movement direction and the second inclination movement direction.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view illustrating a schematic configuration of an entire hand piece of a surgical operating apparatus according to a first embodiment of the present invention;

FIG. 2 is a front view of the hand piece of the surgical operating apparatus according to the first embodiment;

FIG. 3 is a side view illustrating a handle unit of the surgical operating apparatus according to the first embodiment;

FIG. 4 is a perspective view illustrating a disassembled state in which coupling portions of an assemble unit of the surgical operating apparatus according to the first embodiment are detached;

FIG. 5 is a longitudinal sectional view illustrating the hand piece of the surgical operating apparatus according to the first embodiment;

FIG. 6 is a sectional view taken along line VI-VI of FIG. 5;

FIG. 7 is a sectional view taken along line VII-VII of FIG. 6;

FIG. 8 is a sectional view taken along line VIII-VIII of FIG. 6;

FIG. 9 is a sectional view taken along line IX-IX of FIG. 6;

FIG. 10 is a sectional view taken along line X-X of FIG. 6;

FIG. 11 is a sectional view taken along line XI-XI of FIG. 5;

FIG. 12 is a top view illustrating a circuit pattern of a switch substrate of a hand switch in the hand piece of the surgical operating apparatus according to the first embodiment;

FIG. 13 is a longitudinal sectional view illustrating a pressure-receiving portion of the hand switch in the hand piece of the surgical operating apparatus according to the first embodiment in a state that the pressure-receiving portion is maintained in a neutral position;

FIG. 14 is a longitudinal sectional view illustrating the pressure-receiving portion of the hand switch in the hand piece of the surgical operating apparatus according to the first embodiment in a state that the pressure-receiving portion is pressed to a position at the left side with respect to the central position of a pressing surface;

FIG. 15 is a longitudinal sectional view illustrating the pressure-receiving portion of the hand switch in the hand piece of the surgical operating apparatus according to the first embodiment in a state that the pressure-receiving portion is pressed to a position at the right side with respect to the central position of a pressing surface;

FIG. 16 is a perspective view illustrating a surgical operating apparatus according to a second embodiment of the present invention;

FIG. 17 is a longitudinal sectional view illustrating a pressure-receiving portion of a hand switch in a hand piece of the surgical operating apparatus according to the second embodiment in a state that the pressure-receiving portion is maintained in a neutral position;

FIG. 18 is a perspective view illustrating a configuration of a switch operation portion of the hand switch in the hand piece of the surgical operating apparatus according to the second embodiment;

FIG. 19 is a side view illustrating a hand piece of a surgical operating apparatus according to a third embodiment of the present invention;

FIG. 20 is a side view illustrating a hand piece of a surgical operating apparatus according to a fourth embodiment of the present invention; and

FIG. 21 is a perspective view illustrating a hand piece of a surgical operating apparatus according to a fifth embodiment of the present invention.

### DETAILED DESCRIPTION OF THE INVENTION

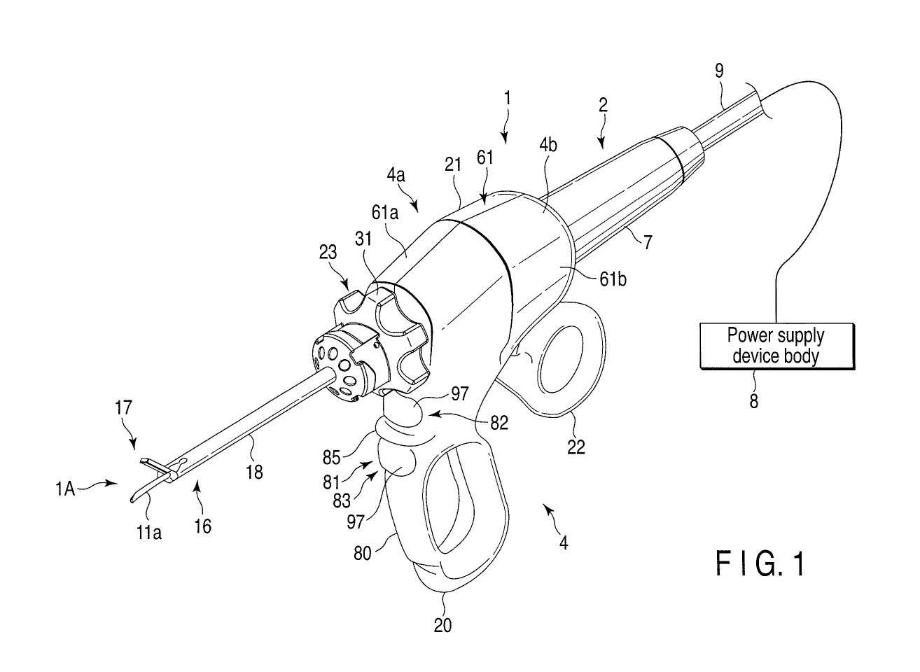

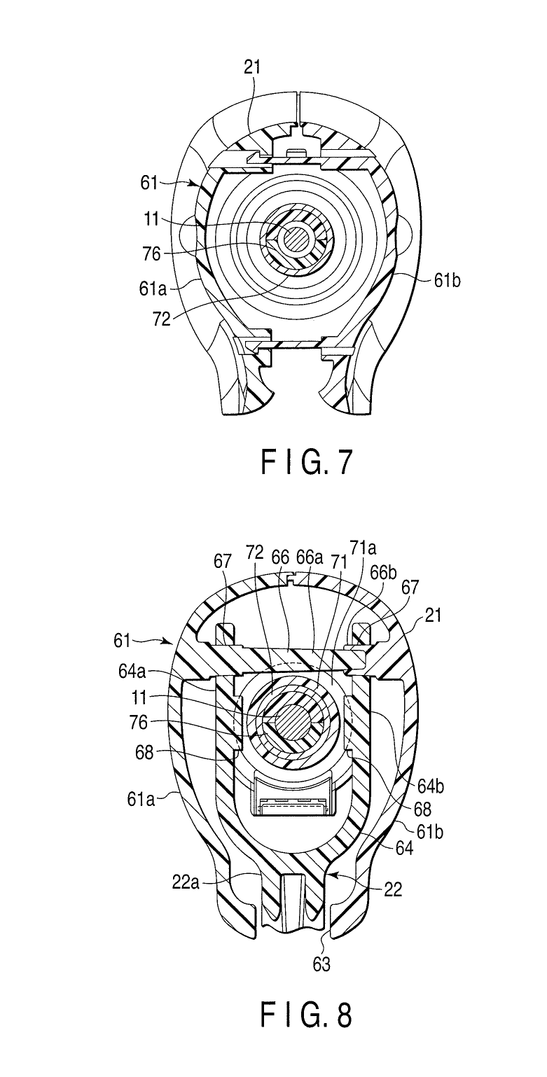

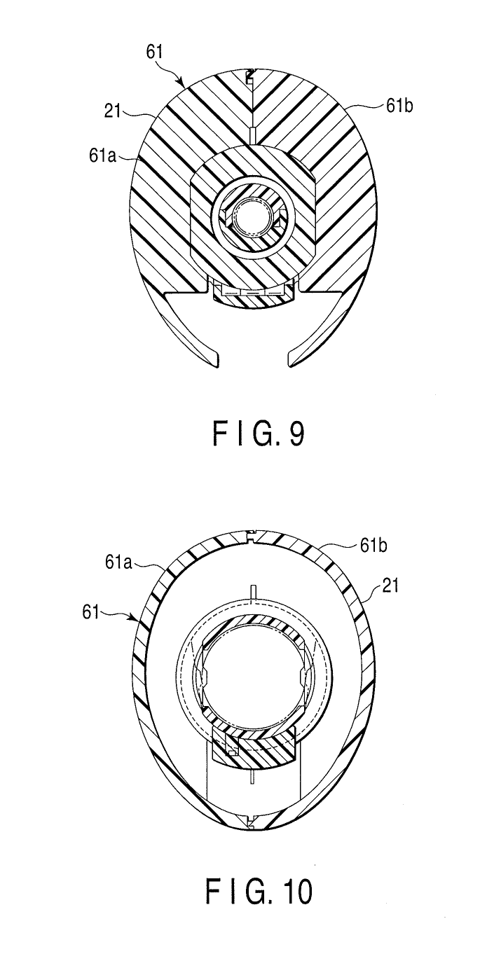

Hereinafter, the first embodiment of the present invention will be described with reference to FIGS. 1 to 15. FIG. 1 is a view illustrating a schematic configuration of an entire hand piece 1 of a surgical operating apparatus according to the present embodiment. The surgical operating apparatus according to the present embodiment is an ultrasonic coagulation-incision treatment apparatus. This ultrasonic coagulation-incision treatment apparatus can perform a treatment such as incision, resection, or coagulation of a living tissue by utilizing ultrasonic waves, as well as a treatment utilizing high-frequency waves.

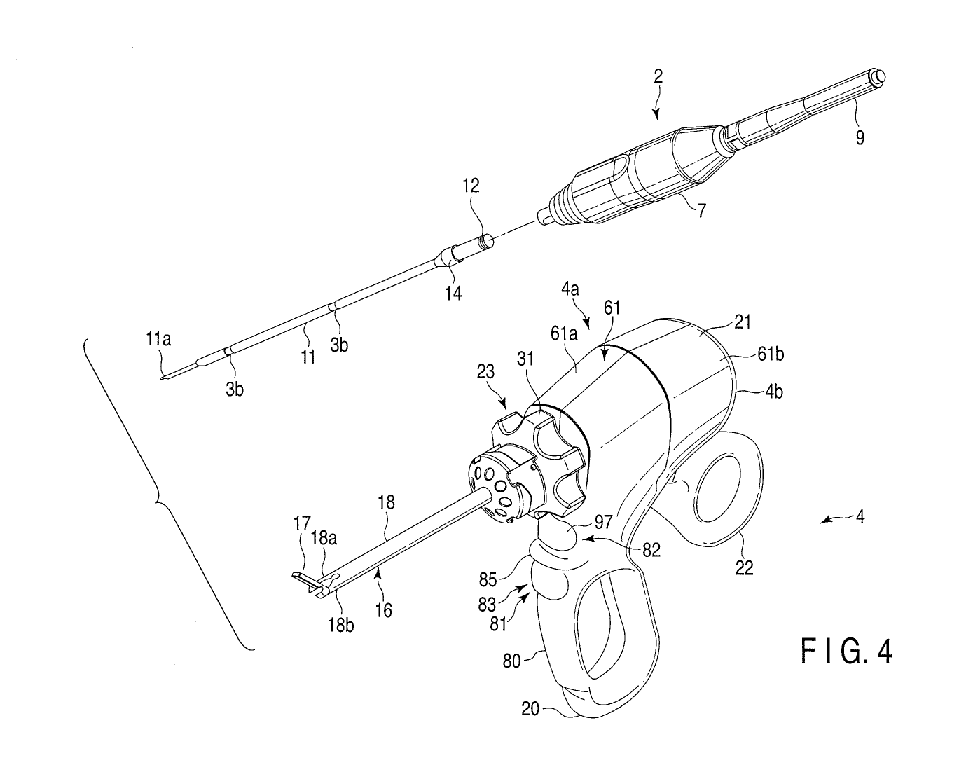

As shown in FIG. 4, the hand piece 1 includes a vibrator unit 2 and a handle unit (handle section) 4. The vibrator unit 2 and handle unit 4 are detachably coupled with each other.

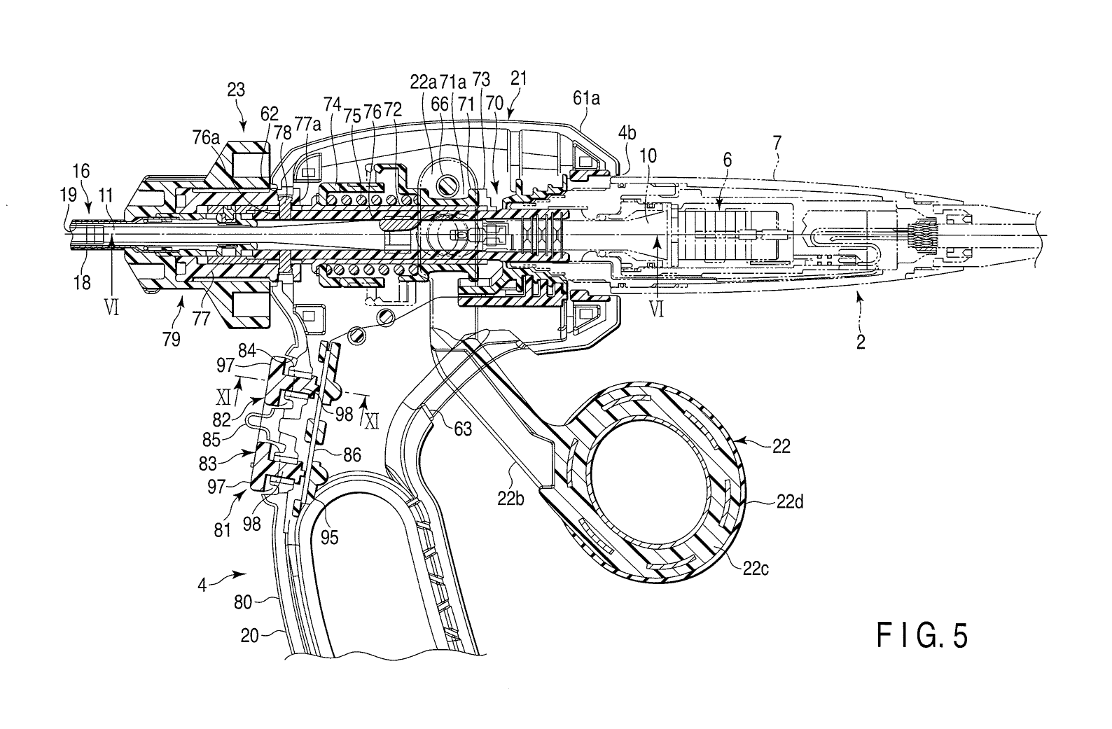

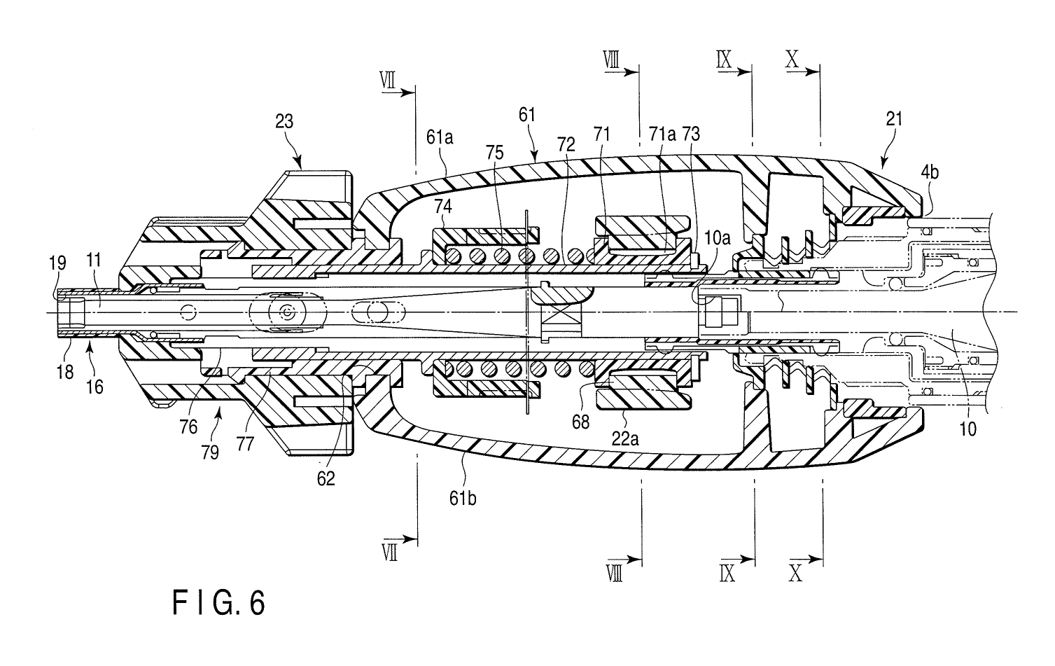

A vibrator 6 (see FIG. 5) is provided in the vibrator unit 2. The vibrator 6 generates ultrasonic vibration by a piezoelectric element which converts an electric current into ultrasonic vibration. An outside of the vibrator 6 is covered with a circular cylindrical vibrator cover 7. Further, a cable 9 configured to supply an electric current which generates ultrasonic vibration from a power supply device body 8 is extended to a proximal end side of the vibrator unit 2. As shown in FIG. 5, a proximal end portion of a horn 10, which increases amplitude of ultrasonic vibration, is coupled to a distal end portion of the ultrasonic vibrator 6 within the vibrator cover 7. A screw hole portion 10a (see FIG. 6), to which a probe is attached, is formed at a distal end portion of the horn 10.

The ultrasonic probe 11 is designed in such a manner that an overall length of the ultrasonic probe 11 is an integral multiple of a half wavelength of the ultrasonic vibration. A screw portion 12 is provided at a proximal end portion of the ultrasonic probe 11. The screw portion 12 is engaged with the screw hole portion 10a of the horn 10. The screw portion 12 of the ultrasonic probe 11 is engaged with the screw hole portion 10a of the horn 10 of the vibrator unit 2. Thereby, the ultrasonic probe 11 and the vibrator 6 are assembled. At this time, a first high-frequency electric path (not shown), through which a high-frequency current is transmitted, is formed in the connected body of the ultrasonic vibrator 6 and the ultrasonic probe 11.

A probe distal end 11a is provided at a distal end portion of the ultrasonic probe 11. The probe distal end 11a is formed in a substantially J-shaped curved form. A flange portion 14 is provided at a position of a most proximal end side node of vibration in an axial direction of the ultrasonic probe 11. Engaging recess portions each having a key groove shape are formed on the outer peripheral surface of the flange portion 14 at three positions in the circumferential direction thereof (not shown).

Further, the probe distal end 11a includes a first electrode section which is one of bipolar electrodes. In the ultrasonic probe 11, the cross-sectional area taken in perpendicular to the axial direction is reduced at several node positions of the vibration, each of which is provided on a midway in the axial direction, so that amplitude necessary for the treatment can be obtained in the treatment with the probe distal end 11a. Each of Rubber rings 3b formed of an elastic member is attached to the ultrasonic probe 11 at a corresponding position of several node positions, each of which is provided on the midway in the axial direction of the ultrasonic probe 11, of the vibration. Further, the rubber rings 3b prevent interference between the ultrasonic probe 11 and a sheath body 16 explained later.

As shown in FIG. 4, the handle unit 4 includes an elongated sheath body 16, a jaw 17 which is provided at a distal end portion of the sheath body 16, and an operation portion 4a provided to a proximal end side of the sheath body 16. A holding tube 21 in a substantially cylindrical shape is provided at the operation portion 4a of the handle unit 4. A vibrator connection portion 4b is formed at a proximal end portion of the holding tube 21.

The sheath body 16 includes an outer sheath 18 made of metal which is an external cylinder, and a drive pipe (drive member) 19 made of metal which is an internal cylinder (inner sheath). The drive pipe 19 is inserted into the outer sheath 18 so as to be movable in the axial direction. An proximal end portion of the outer sheath 18 as well as a rotation operation knob 23, explained later, are integrally attached to the distal end portion of the holding tube 21 in such a manner that the outer sheath 18 can rotate in a direction about an axis of the outer sheath 18.

A pair of right-and-left projection pieces 18a, 18b are provided at the distal end portion of the outer sheath 18 in such a manner that the projection pieces 18a, 18b protrude toward the distal end side. The proximal end part of the jaw 17 is pivotally attached to each of the projection pieces 18a, 18b through a support pin, not shown.

The jaw 17 is formed into a curved shape such as a substantially J-shape corresponding to the curved shape of the probe distal end 11a of the ultrasonic probe 11. When the ultrasonic probe 11 and the handle unit 4 are coupled to each other, the jaw 17 is arranged at a position at which the jaw 17 faces to the probe distal end 11a of the ultrasonic probe 11.

The jaw 17 includes a jaw body (not shown) made of a conductive member such as metal and a grasping member (not shown) to be attached to the jaw body. The grasping member is constituted of an electrode member used in high-frequency treatment and a pad member used in ultrasonic treatment. The electrode member constitutes a second electrode section which is the other of the bipolar electrodes. The pad member is formed of an insulator which is a resin material such as polytetrafluoroethylene and the like.

Further, a distal end portion of the drive pipe 19 is coupled with the jaw 17 via a coupling pin, not shown. The jaw body of the jaw 17 and the drive pipe 19 are electrically connected via the coupling pin.

An outer peripheral surface of the outer sheath 18 is covered with an outer coat formed of an insulating material such as resin. An insulating tube formed of an insulating material is provided to the inner peripheral surface side of the drive pipe 19. A proximal end portion of the insulating tube is extended to a proximal end side part of the sheath body 16. The drive pipe 19 and the ultrasonic probe 11 are electrically insulated by the insulating tube.

The operation portion 4a of the handle unit 4 includes a fixed handle 20 and a movable handle 22 that can pivot with respect to the fixed handle 20. The handle unit 4 includes an operation portion housing 61 formed of a resin material integrally with a holding tube 21 and a fixed handle 20. As shown in FIGS. 6 to 10, the operation portion housing 61 includes two housing components (a left side housing component 61a and a right side housing component 61b) that can be divided into the right and left parts at the axis of the holding tube 21. The left side housing component 61a and the right side housing component 61b are detachably coupled with each other.

At a distal end surface side of the holding tube 21, a knob holding portion 62 configured to hold the rotation operation knob 23 is provided. The rotation operation knob 23 is rotatably coupled with the knob holding portion 62 in the direction about the axis of the outer sheath 18.

A movable handle insertion hole 63 in a long hole shape into which the movable handle 22 is inserted is formed on an upper side portion of a proximal end side surface of the fixed handle 20. As shown in FIG. 5, the movable handle 22 includes an upper arm 22a and a lower arm 22b. The upper arm 22a is extended in a direction perpendicular to the axial direction of the ultrasonic probe 11. The lower arm 22b is bent obliquely downward at a lower end portion of the upper arm 22a in a proximal end direction. A finger insertion ring portion 22c, into which, e.g., a thumb of a user is inserted, is formed at a lower end portion of the lower arm 22b. An elastic ring-shaped finger contact member 22a with a rubber lining is attached to the finger insertion ring portion 22c.

The upper arm 22a is inserted into the operation portion housing 61 through the movable handle insertion hole 63 of the fixed handle 20. As shown in FIG. 8, a U-shaped coupling portion 64 having a substantially U-shape is provided at an upper end portion of the upper arm 22a. The U-shaped coupling portion 64 includes two arms 64a, 64b. The movable handle 22 is attached to a slider member 71 in a state that the slider member 71 of a manipulating force transmission mechanism 70, explained later, is inserted between the two arms 64a, 64b.

A pin insertion hole 67 for a support pin 66 is formed in an upper end portion of each of the arms 64a, 64b. Further, at a lower side part to the pin insertion hole 67 of each of the arms 64a, 64b, an action pin 68 is formed to protrude to the inner side.

The support pin 66 is integrally formed with the operation portion housing 61 in the operation portion housing 61. In this case, a first shaft portion 66a constituting the support pin 66 is formed on the left side housing component 61a. A second shaft portion 66b configured to receive a shaft, into which an end of the first shaft 66a is inserted, is formed in the right side housing component 61b. When the left side housing component 61a and the right side housing component 61b are assembled, the end of the first shaft section 66a is inserted into the second shaft section 66b configured to receive the shaft, so that the first shaft section 66a and the second shaft section 66b are engaged with each other. As a result, the left side housing component 61a and the right side housing component 61b are assembled.

The support pin 66 is inserted into the pin insertion hole 67 of the upper end portion of each of the arms 64a, 64b. Thus, the upper end portion of the movable handle 22 is axially supported by the holding tube 21 via the support pin 66 in a pivotable manner. When the movable handle 22 pivots via the support pin 66, the movable handle 22 is operated to be opened/closed with respect to the fixed handle 20.

As shown in FIG. 5, an operation force transmission mechanism 70 is provided in the holding tube 21 to transmit an operation force applied by the movable handle 22 to the drive pipe 19. The operation force transmission mechanism 70 includes a slider receiving member 72 in a cylindrical shape mainly made of metal and a slider member 71. The slider receiving member 72 is arranged coaxially with the central axis of the holding tube 21, and extended in the same direction as an insertion direction of the ultrasonic probe 11.

A stopper 73 and a spring receiver 74 are provided on an outer peripheral surface of the slider receiving member 72. The stopper 73 is fixed to the outer peripheral surface of a proximal end portion of the slider receiving member 72. The spring receiver 74 is provided on the outer peripheral surface at a distal end side part of the slider receiving member 72 in a protruding manner. The slider member 71 and a coil spring 75 are arranged in the axial direction between the stopper 73 and the spring receiver 74. The stopper 73 is configured to restrict movement of the position the slider member 71 to the proximal end side. A distal end portion of the coil spring 75 is in contact with the spring receiver 74. The coil spring 75 is installed between the spring receiver 74 and the slider member 71 with a constant amount of equipment load.

A ring-shaped engaging groove 71a is formed in a circumferential direction on an outer peripheral surface of the slider member 71. As shown in FIGS. 6 and 8, the action pin 68 of the movable handle 22 is inserted into the engaging groove 71a and engaged therewith. When a user grasps the movable handle 22 to close the movable handle 22 with respect to the fixed handle 20, the action pin 68 pivots about the support pin 66 in accordance with the pivoting movement of the movable handle 22. Concomitantly with the movement of the action pin 68, the slider member 71 moves to a distal direction along the axial direction of the ultrasonic probe 11. At this occasion, the slider receiving member 72 coupled with the slider member 71 via the coil spring 75 also reciprocally moves together with the slider member 71.

A inside of the slider receiving member 72, a tube-shaped probe folder 76 formed of an insulating material is arranged to cover the ultrasonic probe 11. The probe folder 76 is fixed to an inner peripheral surface of the slider receiving member 72. An inner peripheral surface of the probe folder 76 and the outer peripheral surface of the ultrasonic probe 11 are fixed via a rotation stop section, not shown. For example, the rotation stop section includes a key groove formed on one of the inner peripheral surface of the probe folder 76 and the outer peripheral surface of the ultrasonic probe 11, and a key formed on the other of the inner peripheral surface of the probe folder 76 and the outer peripheral surface of the ultrasonic probe 11. In this configuration, the probe folder 76 can move in the axial direction with respect to the ultrasonic probe 11, and the probe folder 76 is restricted such that the probe folder 76 cannot relatively rotate in the direction about the axis with respect to the ultrasonic probe 11.

A proximal end portion of the drive pipe 19 is fixed to a distal end portion of the probe folder 76. Therefore, the operation force of the movable handle 22 is transmitted to the drive pipe 19 via the slider member 71, the coil spring 75, the slider receiving member 72, and the probe folder 76, and the drive pipe 19 moves in the axial direction. When the drive pipe 19 moves, the jaw 17 pivots via the support pin.

As described above, when the drive pipe 19 reciprocally moves in the axial direction, the drive force of the drive pipe 19 is transmitted to the jaw 17 via the coupling pin. Thereby, the jaw 17 is driven to pivot about the support pin. At this occasion, when the drive pipe 19 is operated and pulled to the proximal end side, the jaw 17 is driven to move about the support pin in a direction away from the probe distal end 11a (open position). On the contrary, when the drive pipe 19 is operated and pushed out forward, the jaw 17 is driven to move about the support pin in a direction toward the probe distal end 11a (closed position). When the jaw 17 is operated and pivoted to the closed position, a living tissue is sandwiched between the jaw 17 and the probe distal end 11a of the ultrasonic probe 11.

In the hand piece 1, the treatment section 1A is constituted by the jaw 17, and the probe distal end 11a of the ultrasonic probe 11. In the treatment section 1A, a plurality of, for example, in present embodiment, two treatment functions (a first treatment function and a second treatment function) can be selected. For example, the first treatment function is set as a function of simultaneously outputting an ultrasonic treatment output and a high-frequency treatment output. The second treatment function is set as a function of outputting only the high-frequency treatment output alone.

It should be noted that the first treatment function and the second treatment function of the treatment section 1A are not limited to the above configurations. For example, a configuration may be employed in which the first treatment function is set as a function of outputting the ultrasonic treatment output with a maximum output state, and the second treatment function is set as a function of outputting the ultrasonic treatment output with an output state set in advance which is lower than the maximum output state.

A knob receiving member 77 in a cylindrical shape is arranged on an inner peripheral surface of the rotation operation knob 23. The inner peripheral surface of the rotation operation knob 23 and an outer peripheral surface of the knob receiving member 77 are fixed via a rotation stop section, not shown. The rotation stop section includes a key groove formed on one of the inner peripheral surface of the rotation operation knob 23 and the outer peripheral surface of knob receiving member 77, and a key formed on the other of the inner peripheral surface of the rotation operation knob 23 and the outer peripheral surface of knob receiving member 77. In this configuration, the rotation operation knob 23 and the knob receiving member 77 are restricted such that the rotation operation knob 23 and the knob receiving member 77 cannot relatively rotate in the direction about the axis.

A proximal end portion of the knob receiving member 77 is coupled with the knob holding portion 62 provided at the distal end portion of the holding tube 21 in such a manner that the knob receiving member 77 can rotate about the direction about the axis of the ultrasonic probe 11. Thus, the rotation operation knob 23 is coupled with the knob holding portion 62 of the holding tube 21 in such a manner that the rotation operation knob 23 can rotate in the direction about the axis of the ultrasonic probe 11.

The distal end portion of the probe folder 76 is arranged to the inside of the knob receiving member 77. A pin insertion hole 77a extended in a radial direction is made in the knob receiving member 77, and a pin insertion hole 76a extended in the radial direction is made in the probe folder 76. A coupling pin 78 is inserted into the pin insertion hole 77a of the knob receiving member 77 and the pin insertion hole 76a of the probe folder 76. The knob receiving member 77 and the probe folder 76 are fixed by the coupling pin 78, and are restricted in such a state that they cannot relatively rotate in the direction about the axis with respect to each other.

When the rotation operation knob 23 is operated and rotated, the knob receiving member 77 and the rotation operation knob 23 rotate together. At this occasion, a rotation of the knob receiving member 77 is transmitted to the probe folder 76 via the coupling pin 78. Further, a rotation of the probe folder 76 is transmitted to the ultrasonic probe 11 via the rotation stop section, explained above, disposed between the probe folder 76 and the ultrasonic probe 11. Therefore, the rotation of the rotation operation knob 23 is transmitted to the ultrasonic probe 11 via the knob receiving member 77, the coupling pin 78, and the probe folder 76 in this order, so that the ultrasonic probe 11 as well as the rotation operation knob 23 rotates together. In other words, a rotation torque transmission section 79 is constituted by the rotation operation knob 23, the knob receiving member 77, the coupling pin 78, and the probe folder 76, which is configured to transmit a rotation torque of the rotation operation knob 23 in the direction about the axis of the ultrasonic probe 11 to the ultrasonic probe 11.

In addition, when the rotation operation knob 23 is operated and rotated, the outer sheath 18 as well as the rotation operation knob 23 rotates together in the direction of the axis about the outer sheath 18 with respect to the holding tube 21. Thus, when the rotation operation knob 23 is operated and rotated, the ultrasonic probe 11 and the outer sheath 18 of the sheath body 16 simultaneously rotate in the direction about the axis of the outer sheath 18. Accordingly, the probe distal end 11a of the ultrasonic probe 11 and the jaw 17 are operated and rotated simultaneously in the same direction.

The operation portion housing 61 includes a plural-fingers insertion ring portion 80 at a lower end portion of the fixed handle 20. For example, a plurality of fingers of a user other than a thumb are inserted into the plural-fingers insertion ring portion 80. As shown in FIG. 5, the fixed handle 20 according to the present embodiment includes a switch holding section 81 disposed between the plural-fingers insertion ring portion 80 and the holding tube 21.

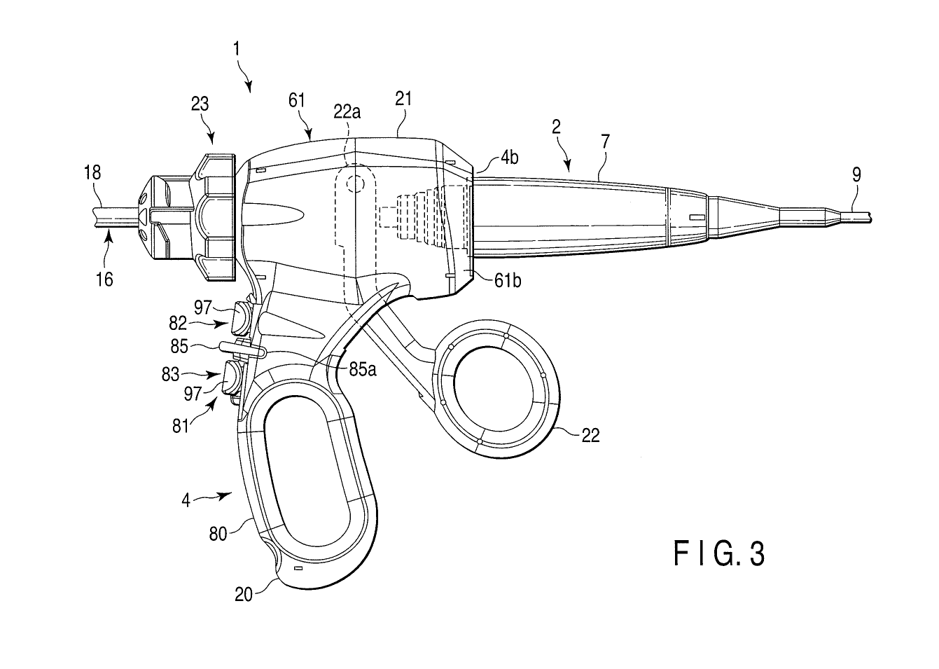

As shown in FIG. 3, the switch holding section 81 includes a switch attachment surface 84 which is provided on a distal end surface of the fixed handle 20, and to which a plurality of, for example, in the present embodiment, two hand switches (a first switch 82 and a second switch 83) are attached. Each of these first switch 82 and second switch 83 is a switch configured to select a treatment function of the treatment section 1A of the hand piece 1.

In the switch holding section 81, the first switch 82 and the second switch 83 are arranged in the vertical direction. Further, a protuberance section 85 is formed between the first switch 82 and the second switch 83 in a protruding manner to the distal end side. The user places a finger on the protuberance section 85.

The first switch 82 is arranged to the upper side of the protuberance section 85. The first switch 82 is set as a switch configured to select the first treatment function which is frequently used among the plural treatment functions.

The second switch 83 is arranged to the lower side of the protuberance section 85. The second switch 83 is set as a switch configured to select the second treatment function which is different from the first treatment function among the plural treatment functions. For example, the first switch 82 is set as a switch button used in coagulation and incision, and the second switch 83 is set as a switch button used in coagulation.

The protuberance section 85 is set in such a manner that the height of the protrusion thereof from the switch attachment surface 84 is more than those of the first switch 82 and the second switch 83. The protuberance section 85 includes an extension portion 85a extended from the switch attachment surface 84 of the fixed handle 20 toward both side surfaces of the fixed handle 20.

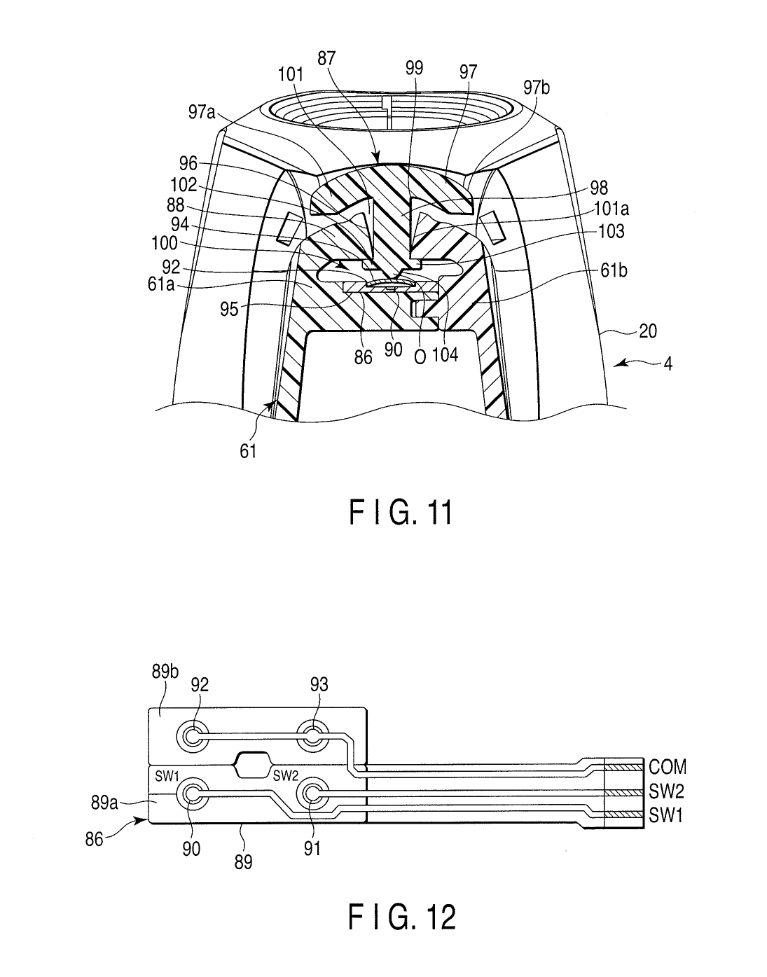

FIG. 11 is a figure illustrating a schematic configuration of the first switch 82 of the hand piece 1. It should be noted that the second switch 83 has the same configuration as the first switch 82. For this reason, only the configuration of the first switch 82 will be hereinafter explained. Description about the configuration of the second switch 83 will not be made.

The first switch 82 includes a switch substrate 86, a switch operation portion 87, and a support portion 88. The switch substrate 86 includes a transmission section, not shown, configured to transmit a signal of driving the apparatus. As shown in FIG. 12, the switch substrate 86 includes a substrate sheet 89 formed of a flexible substrate and the like. The substrate sheet 89 includes a sheet body 89a and a bent portion 89b. The sheet body 89a is provided with a first switch contact point 90 of the first switch 82 and a second switch contact point 91 of the second switch 83. The bent section 89b is provided with a first common contact point 92 of the first switch 82 and a second common contact point 93 of the second switch 83. When the substrate sheet 89 is used, the bent section 89b is bent with respect to the sheet body 89a. Further, the switch substrate 86 includes a dome-shaped metal dome (urging member) 94 made of a metal material provided for each of the first switch 82 and the second switch 83. With the metal dome 94, the switch contact point 90 of the switch substrate 86 is maintained in an open state. When the switch operation portion 87, explained later, is operated, the metal dome 94 is elastically deformed and squashed, whereby the switch contact point 90 is closed. As a result, the first switch contact point 90 of the first switch 82 and the first common contact point 92 are connected, and the transmission section of the first switch 82 of the switch substrate 86 transmits the signal of driving the apparatus.

The operation portion housing 61 is provided with a substrate holding section 95 configured to hold the switch substrate 86 and a switch operation portion holding section 96 configured to hold the switch operation portion 87. The substrate holding section 95 holds the switch substrate 86 at position corresponding to the first switch 82 and the second switch 83 in the operation portion housing 61.

The switch operation portion 87 includes a pressure-receiving portion 97 configured to be pressed by the user. The pressure-receiving portion 97 is supported by the support portion 88 in such a manner that the pressure-receiving portion 97 can move about an inclination movement center O in the operation portion housing 61 to incline with respect to the operation portion housing 61. During pressing operation in which the pressure-receiving portion 97 is pressed, the support portion 88 supports the pressure-receiving portion 97 in such a state that the pressure-receiving portion 97 can be pressed in a direction where the inclination movement center O of the pressure-receiving portion 97 is arranged against a force applied by the metal dome 94. On the other hand, during non-pressing operation of the pressure-receiving portion 97, the support portion 88 holds the switch operation portion 87 at a position where the switch contact point 90 is held in an open state.

The switch operation unit 87 includes a shaft section 98. At one end portion of the shaft section 98 (end portion on a side of the pressure-receiving unit 97), a first coupling section 99 coupling with the pressure-receiving portion 97 is provided. At the other end portion of the shaft section 98 (end portion on a side of the switch contact point 90), a second coupling section 100 coupling with the operation portion housing 61 is provided.

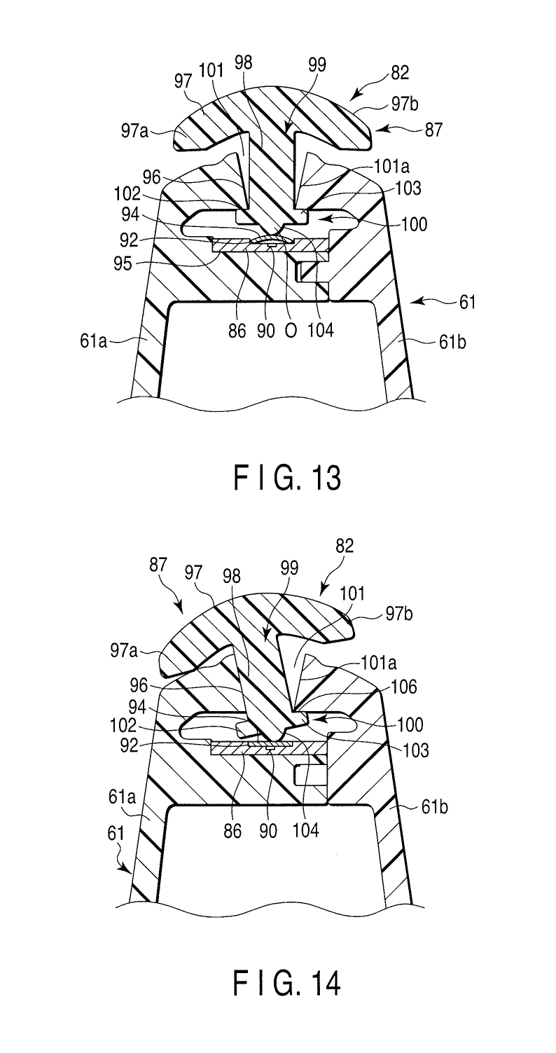

The support portion 88 includes a clearance formation section 101 configured to form a clearance between the support portion 88 and a portion of the shaft section 98 of the switch operation portion 87 on a side of the first coupling section 99. With the clearance formed by the clearance formation section 101, the pressure-receiving portion 97 can move about the inclination movement center O of the pressure-receiving portion 97 in inclination movement directions. The clearance formation section 101 includes an inclination surface 101a whose size of cross sectional area of the clearance gradually increases from a switch contact point 90 side to a pressure-receiving portion 97 side. FIG. 13 is a figure illustrating the pressure-receiving portion 97 of the first switch 82 maintained in the non-pressed state when the pressure-receiving portion 97 and the shaft section 98 are maintained in a neutral position without inclination. In this case, the pressure-receiving portion 97 and the shaft section 98 are restricted such that the pressure-receiving portion 97 and the shaft section 98 are inclined from the neutral position to inclination states in two directions, i.e., a first inclination movement direction in which the pressure-receiving portion 97 and the shaft section 98 are inclined in the left direction in FIG. 13 and a second inclination movement direction in which the pressure-receiving portion 97 and the shaft section 98 are inclined in the right direction in FIG. 13. As shown in FIG. 5, inclination in a direction perpendicular to the inclination movement directions of the pressure-receiving portion 97 and the shaft section 98 is restricted by wall surfaces of the operation portion housing 61 configured to come into contact with the shaft section 98. For this reason, the inclination movement directions of the pressure-receiving portion 97 are restricted to only two directions, i.e., a first inclination movement direction in which the pressure-receiving portion 97 inclines from the neutral position to the left side in FIG. 13 and a second inclination movement direction in which the pressure-receiving portion 97 inclines from the neutral position to the right side in FIG. 13.

The pressure-receiving portion 97 of the first switch 82 is a push button formed in a long arc shape, in which the pressing surface is formed along two directions, i.e., the first inclination movement direction and the second inclination movement direction. A first coupling section 99, serving as a coupling section with the shaft section 98, is provided at a central position of the arc-shaped pressing surface of the pressure-receiving portion 97. The pressure-receiving portion 97 includes a left side extension section (first inclination movement direction extension section) 97a extended from the first coupling section 99 to the left side in FIG. 13 (first inclination movement direction) and a right side extension section (second inclination movement direction extension section) 97b extended from the first coupling section 99 to the right side in FIG. 13 (second inclination movement direction).

The second coupling section 100 includes a first protrusion 102 protruding in the first inclination movement direction, a second protrusion 103 protruding in the second inclination movement direction, and a switch contact section 104 configured to be in contact with the metal dome 94 of the switch substrate 86.

When the user straightly presses the pressure-receiving portion 97 of the first switch 82 from the neutral position of FIG. 13, the switch contact section 104 presses the metal dome 94 of the switch substrate 86. Accordingly, the metal dome 94 of the first switch 82 is elastically deformed and squashed. As a result, the first switch contact point 90 of the first switch 82 and the first common contact point 92 are brought into contact, whereby the switch contact point 90 of the first switch 82 is closed.

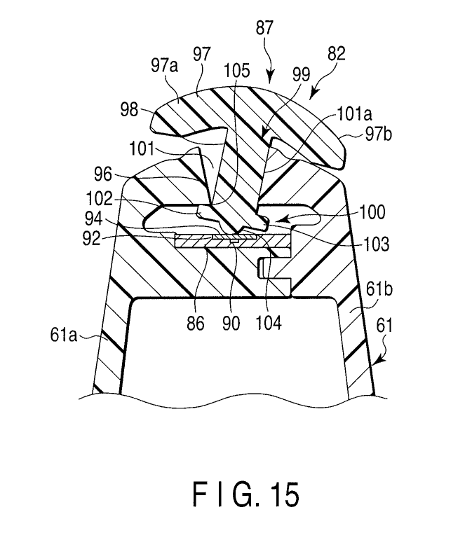

As shown in FIG. 15, when the right side extension section (second inclination movement direction extension section) 97b of the pressure-receiving portion 97 is pressed, the pressure-receiving portion 97 of the first switch 82 is inclined and moved in the second inclination movement direction. At this occasion, a first inclination movement support point 105 is formed at a contact section between the first protrusion 102 and the inner surface of the operation portion housing 61. When the shaft section 98 is inclined and moved about the first inclination movement support point 105, the switch contact section 104 presses the metal dome 94 of the switch substrate 86. Accordingly, the metal dome 94 of the first switch 82 is elastically deformed and squashed. As a result, the first switch contact point 90 of the first switch 82 and the first common contact point 92 are brought into contact, whereby the switch contact point 90 of the first switch 82 is closed.

On the other hand, as shown in FIG. 14, when the left side extension section (first inclination movement direction extension section) 97a of the pressure-receiving portion 97 is pressed, the pressure-receiving portion 97 of the first switch 82 is inclined and moved in the first inclination movement direction. At this occasion, a second inclination movement support point 106 is formed at a contact section between the second protrusion 103 and the inner surface of the operation portion housing 61. When the shaft section 98 is inclined and moved about the second inclination movement support point 106, the switch contact section 104 presses the metal dome 94 of the switch substrate 86. Accordingly, the metal dome 94 of the first switch 82 is elastically deformed and squashed. As a result, the first switch contact point 90 of the first switch 82 and the first common contact point 92 are brought into contact, whereby the switch contact point 90 of the first switch 82 is closed.

It should be noted that the second switch 83 is operated in the same manner as the first switch 82. Accordingly, the metal dome 94 of the second switch 83 is elastically deformed and squashed. As a result, the second switch contact point 91 of the second switch 82 and the second common contact point 93 are connected, whereby the switch contact point 91 of the second switch 83 is closed. Accordingly, the transmission section of the second switch 83 of the switch substrate 86 transmits the signal of driving the apparatus.

It should be noted that a rubber cover which covers the entire outer peripheral surface of the pressure-receiving portion 97 of the first switch 82 and a rubber cover which covers the entire outer peripheral surface of the pressure-receiving portion 97 of the second switch 83 may be provided on the operation portion housing 61. This makes a waterproof sealing structure in the first switch 82 and the second switch 83.

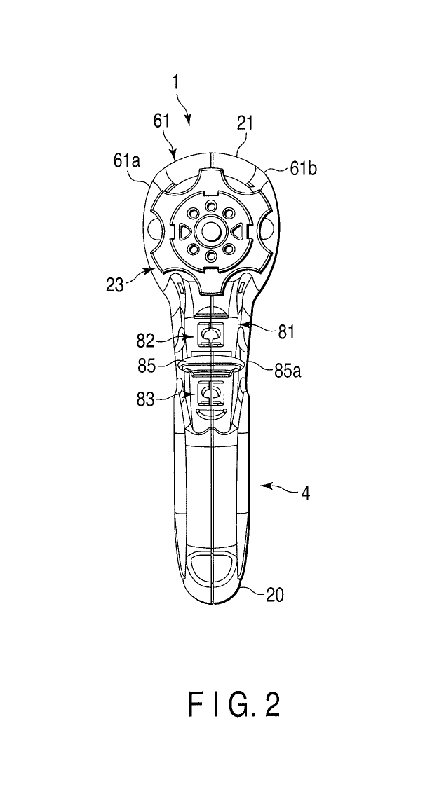

Subsequently, the function of the present embodiment will be explained. As shown in FIG. 2, in the hand piece 1 of the surgical operating apparatus according to the present embodiment, two units, i.e., the vibrator unit 2 and the handle unit 4, are detachably coupled. When the hand piece 1 is used, the vibrator unit 2 and the handle unit 4 are coupled to each other. As a result of this, in the connected body of the vibrator unit 2 and the ultrasonic probe 11, a first high-frequency electric pathway through which a high-frequency current is configured to be transmitted is formed.

On the other hand, in the hand piece 1, a second high-frequency electric pathway through which a high-frequency current is configured to be transmitted to the electrode member of the jaw 17 is formed. When the high-frequency current is transmitted to the electrode member of the jaw 17, the second electrode section of the bipolar electrode is formed. As described above, the hand piece 1 is assembled.

When the hand piece 1 is used, the movable handle 22 is operated to be opened or closed with respect to the fixed handle 20. The drive pipe 19 is moved in the axial direction in accordance with the operation of the movable handle 22, and the jaw 17 is driven to be opened or closed with respect to the probe distal end 11a of the ultrasonic probe 11 in accordance with the advancing/retreating operation of the drive pipe 19 in the axial direction. In this case, when the movable handle 22 is operated to be closed with respect to the fixed handle 20, the drive pipe 19 is pushed to the distal end side in accordance with the operation of the movable handle 22. The jaw 17 is driven in a direction in which the jaw 17 is made closer to the probe distal end 11a of the ultrasonic probe 11 (closed position) in accordance with the pushing operation of the drive pipe 19. When the jaw 17 is operated and pivoted to the closed position, a living tissue is sandwiched between the jaw 17 and the probe distal end 11a of the ultrasonic probe 11.

At this state, any one of the first switch 82 and the second switch 83 of the fixed handle 20 is selectively pushed. When the first switch 82 is pushed, a drive current is passed through the ultrasonic vibrator 6 and the ultrasonic vibrator 6 is driven, and simultaneously the high-frequency current is turned on. At this time, the ultrasonic vibration from the ultrasonic vibrator 6 is transmitted to the probe distal end 11a through a vibration transmission member 11. As a result of this, it is possible to perform a treatment such as incision, resection, and the like of the living tissue by utilizing the ultrasonic waves together with the application of the high-frequency waves. It should be noted that it is also possible to perform a coagulation treatment of the living tissue by utilizing the ultrasonic waves.

When the first switch 82 is pressed, and the pressure-receiving portion 97 of the first switch 82 is straightly pressed from the neutral position at which the pressure-receiving portion 97 is not inclined as shown in FIG. 13, the switch contact section 104 presses the metal dome 94 of the switch substrate 86. Accordingly, the metal dome 94 of the first switch 82 is elastically deformed and squashed. As a result, the first switch contact point 90 of the first switch 82 and the first common contact point 92 are brought into contact, whereby the switch contact point 90 of the first switch 82 is closed. Accordingly, the transmission section of the first switch 82 of the switch substrate 86 transmits the signal of driving the apparatus.

As shown in FIG. 15, when the right side extension section (second inclination movement direction extension section) 97b of the pressure-receiving portion 97 is pressed, the pressure-receiving portion 97 of the first switch 82 is inclined and moved in the second inclination movement direction. At this occasion, a first inclination movement support point 105 is formed at a contact section between the first protrusion 102 and the inner surface of the operation portion housing 61. When the shaft section 98 is inclined and moved about the first inclination movement support point 105, the switch contact section 104 presses the metal dome 94 of the switch substrate 86. Accordingly, the metal dome 94 of the first switch 82 is elastically deformed and squashed. As a result, the first switch contact point 90 of the first switch 82 and the first common contact point 92 are brought into contact, whereby the switch contact point 90 of the first switch 82 is closed. Accordingly, the transmission section of the first switch 82 of the switch substrate 86 transmits the signal of driving the apparatus

On the other hand, as shown in FIG. 14, when the left side extension section (first inclination movement direction extension section) 97a of the pressure-receiving portion 97 is pressed, the pressure-receiving portion 97 of the first switch 82 is inclined and moved in the first inclination movement direction. At this occasion, a second inclination movement support point 106 is formed at a contact section between the second protrusion 103 and the inner surface of the operation portion housing 61. When the shaft section 98 is inclined and moved about the second inclination movement support point 106, the switch contact section 104 presses the metal dome 94 of the switch substrate 86. Accordingly, the metal dome 94 of the first switch 82 is elastically deformed and squashed. As a result, the first switch contact point 90 of the first switch 82 and the first common contact point 92 are brought into contact, whereby the switch contact point 90 of the first switch 82 is closed. Accordingly, the transmission section of the first switch 82 of the switch substrate 86 transmits the signal of driving the apparatus.

Therefore, in the present embodiment, the user can perform the switch operation which is adapted to cause the transmission section of the first switch 82 of the switch substrate 86 to transmit the signal of driving the apparatus no matter at which position of the arc-shaped pressing surface of the pressure-receiving portion 97 of the first switch 82 the user presses the pressure-receiving portion 97.

When the user cancels pressing of the pressure-receiving portion 97 of the first switch 82, the metal dome 94 of the first switch 82 elastically recovers back to its original dome shape, so that the connection between the first switch contact point 90 of the first switch 82 and the first common contact point 92 is disconnected, whereby the switch contact point 90 of the first switch 82 is opened.

When the second switch 83 is pressed, the first high-frequency electric path through which the high-frequency current flows to the probe distal end 11a of the ultrasonic probe 11 and the second high-frequency electric path through which the high-frequency current flows to the jaw body of the jaw 17 are conducted. Accordingly, two bipolar electrodes used in high-frequency treatment are constituted by the probe distal end 11a of the ultrasonic probe 11 and the jaw body. By causing a high-frequency current to flow between the two bipolar electrodes that are the probe distal end 11a of the ultrasonic probe 11 and the jaw 17, a high-frequency treatment by the bipolar electrodes can be applied to the living tissue between the jaw 17 and the probe distal end 11a of the ultrasonic probe 11.

It should be noted that the second switch 83 is operated in the same manner as the first switch 82. Accordingly, the metal dome 94 of the second switch 83 is elastically deformed and squashed. As a result, the second switch contact point 91 of the second switch 82 and the second common contact point 93 are connected, whereby the switch contact point 91 of the second switch 83 is closed. Accordingly, the transmission section of the second switch 83 of the switch substrate 86 transmits the signal of driving the apparatus.

When the movable handle 22 is operated to be opened with respect to the fixed handle 20, the drive pipe 19 is pulled to the proximal end side in accordance with the operation of opening the movable handle 22. When the drive pipe 19 is pulled, the jaw 17 is driven to move in a direction away from the probe distal end 11a of the ultrasonic probe 11 (open position).

Therefore, the hand piece 1 having the above configuration achieves the following effects. That is, the hand piece 1 of the present embodiment is provided with the support portion 88 configured to support the pressure-receiving portion 97 of the first switch 82 in such a manner that the pressure-receiving portion 97 of the first switch 82 can incline about the inclination movement center O in the operation portion housing 61. Accordingly, when the user presses a part away from the central position of the arc-shaped pressing surface of the pressure-receiving portion 97 of the first switch 82, the pressure-receiving portion 97 can be inclined and moved about the inclination movement center O in the operation portion housing 61. When the pressure-receiving portion 97 is inclined and moved with respect to the operation portion housing 61, the pressing surface of the pressure-receiving portion 97 of the first switch 82 is moved, so that the operator can press the pressing surface of the pressure-receiving portion 97 while the operator relaxes the fingers to easily press the pressure-receiving portion 97. In other words, the pressure-receiving portion 97 can be inclined and moved within the movable range of the finger of the user. As a result, unlike a conventional medical apparatus, after the operator moves the finger to the position where the switch is provided so as to press the switch, the operator does not have to straightly press an activation rod of the switch in an axial direction of the switch while the operator maintains the posture of the finger without changing the posture of the finger so as to easily press the switch. This improves the ease of operation and the feeling of pressing operation when the operator operates the first switch 82.

In addition, in the present embodiment, the pressure-receiving portion 97 of each of the first switch 82 and the second switch 83 is a push button whose pressing surface is formed in a long arc shape along two directions, i.e., the first direction and the second direction. In this configuration, no matter at which position of the arc-shaped pressing surface the pressure-receiving portion 97 of each of the first switch 82 and the second switch 83 is pressed, the user can perform the switch operation of causing the transmission section of the first switch 82 or the second switch 83 of the switch substrate 86 to transmit the signal of driving the apparatus. For this reason, even when the user pushes a position of the pressure-receiving portion 97 away from the central position of the pressure-receiving portion 97 during operation of the first switch 82 or the second switch 83, the switch can be operated. Therefore, the user's ease of operation can be improved, and the switch can be operated stably.

In addition, in the present embodiment, when the pressure-receiving portion 97 of the first switch 82 or the second switch 83 is pressed, the metal dome 94 of the first switch 82 or the second switch 83 is elastically deformed and squashed, whereby a click sound is generated. Therefore, the operator can feel the click, which reliably allows the operator to confirm the switch operation of the first switch 82 or the second switch 83.

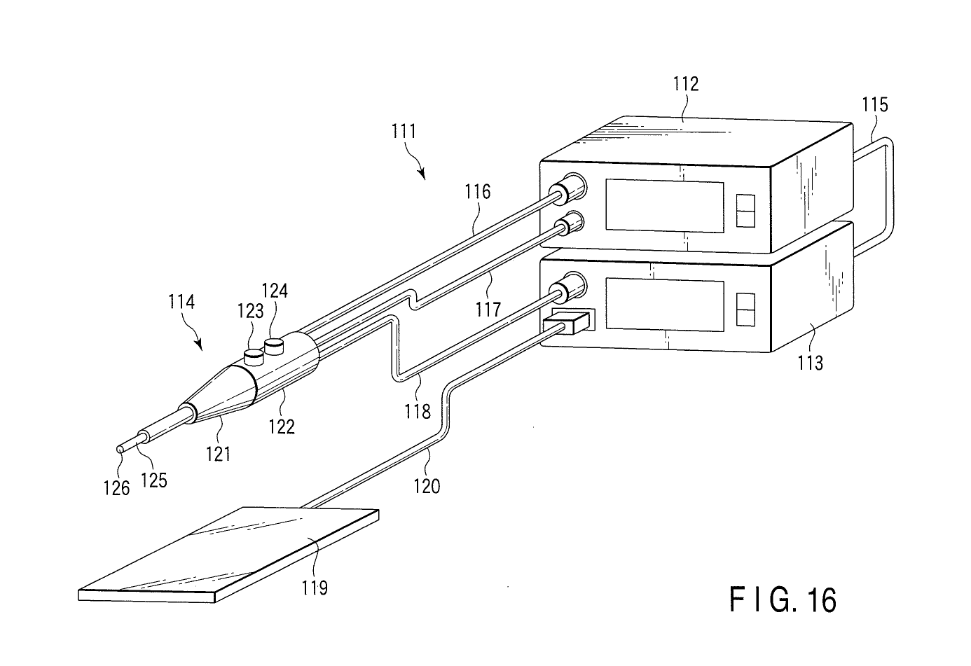

FIGS. 16 to 18 are figures illustrating the second embodiment of the present invention. In the present embodiment, a surgical apparatus 111 having a configuration different from that of the surgical operating apparatus according to the first embodiment (see FIGS. 1 to 15) is used as an apparatus.

The surgical apparatus 111 mainly includes an ultrasonic drive device 112, a high-frequency drive device 113, and a hand piece 114. The ultrasonic drive device 112 and the high frequency drive device 113 are connected to each other by way of a communication cable 115.

The hand piece 114 is connected to the ultrasonic drive device 112 by way of an output connection cable 116 and an SW connection cable 117 via a connector. The hand piece 114 is connected to the high frequency drive device 113 by way of an output connection cable 118 via a connector. A counter electrode plate 119 is connected to the high frequency drive device 113 by way of a connection cable 120 via a connector.

The hand piece 114 includes a sheath body 121 provided at a distal end side part and a substantially cylindrical-shaped operation portion housing 122 provided at a proximal end side part. The operation portion housing 122 and the sheath body 121 are fitted into each other. The operation portion housing 122 includes two housing components (a left side housing component 122a and a right side housing component 122b) that can be divided into the right-and-left parts. The left side housing component 122a and the right side housing component 122b are detachably coupled with each other.

An ultrasonic vibrator (e.g., bolt-clamped Langevin type vibrator), not shown, is provided in the operation portion housing 122 configured to generate ultrasonic vibration. Electric power used in driving an ultrasonic wave is supplied from the ultrasonic drive device 112 to the ultrasonic vibrator via the output connection cable 116.

A probe 125 configured to transmit ultrasonic vibration is screwed to a distal end side of the ultrasonic vibrator. A treatment section 126 is arranged at a distal end portion of the probe 125 in order to treat living tissue.

Further, the operation portion housing 122 is provided with a first switch 123 and a second switch 124 as a hand switch. The first switch 123 and the second switch 124 are mounted on a switch substrate 131 which is an electric circuit substrate accommodated in the operation portion housing 122. A lead in the SW connection cable 117 is connected to a connection terminal of the switch substrate 131 typically by soldering. The SW connection cable 117 is extended externally from a proximal end side of the operation portion housing 122 and is connected to the ultrasonic drive device 112 via a connector. With this arrangement, the electric signals of the first switch 123 and the second switch 124 can be transmitted to the ultrasonic drive device 112 by way of the SW connection cable 117.

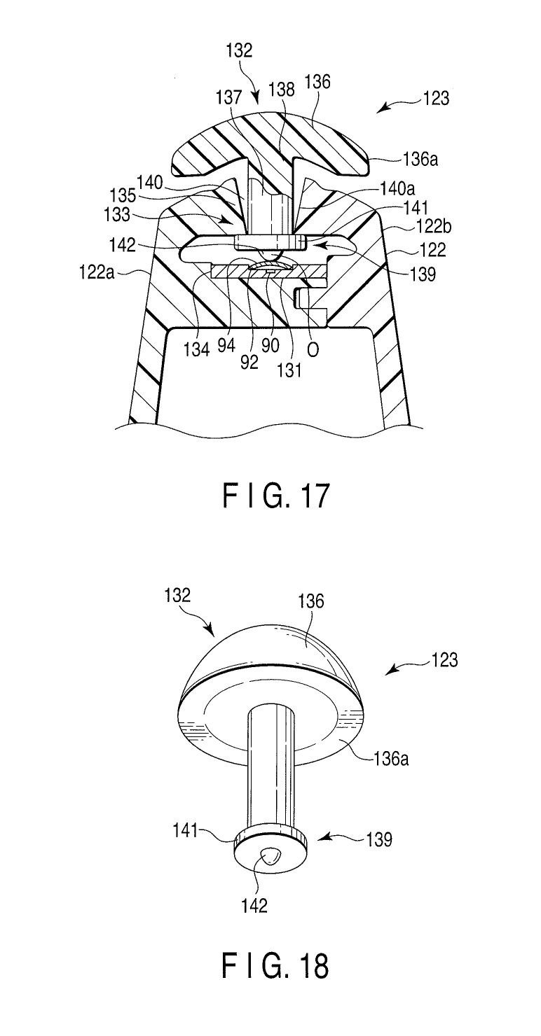

FIG. 17 is a figure illustrating a schematic configuration of the first switch 123 of the hand piece 114. It should be noted that the second switch 124 has the same configuration as the first switch 123. Therefore, for this reason, only the configuration of the first switch 123 will be hereinafter explained. Description about the configuration of the second switch 124 will not be made.

The first switch 124 includes a switch substrate 131, a switch operation portion 132, and a support portion 133. Since the switch substrate 131 has the same configuration as the switch substrate 86 according to the first embodiment, the same parts as those of the switch substrate 86 of the first embodiment are denoted with the same reference numerals and description thereof will not be made.

The operation portion housing 122 is provided with a substrate holding section 134 configured to hold the switch substrate 131 and a switch operation portion holding section 135 configured to hold the switch operation portion 132. The substrate holding section 134 holds the switch substrate 131 at position corresponding to the first switch 123 and the second switch 124 in the operation portion housing 122.

The switch operation portion 132 includes a pressure-receiving portion 136 configured to be pressed by the user. The pressure-receiving portion 132 is supported by the support portion 133 in such a manner that the pressure-receiving portion 132 can move about an inclination movement center O in the operation portion housing 122 to incline with respect to the operation portion housing 122. During pressing operation in which the pressure-receiving portion 136 is pressed, the support portion 133 supports the pressure-receiving portion 136 in such a state that the pressure-receiving portion 136 can be pressed in a direction where the inclination movement center O of the pressure-receiving portion 136 is arranged against the force applied by the metal dome 94. On the other hand, during non-pressing operation of the pressure-receiving portion 136, the support portion 133 holds the switch operation portion 132 at a position where the switch contact point 90 is held in an open state. The switch operation portion 132 includes a shaft section 137. At one end portion of the shaft section 137 (end portion on the side of the pressure-receiving portion 136), a first coupling section 138 coupling with the pressure-receiving portion 136 is provided. At the other end portion of the shaft section 137 (end portion on the side of the switch contact point 90), a second coupling section 139 coupling with the operation portion housing 122 is provided.

The support portion 133 includes a clearance formation section 140 configured to form a clearance between the support portion 133 and a first coupling section 138 side part of the shaft section 137 of the switch operation portion 132. With the clearance formed by the clearance formation section 140, the pressure-receiving portion 136 can move about the inclination movement center O of the pressure-receiving portion 136 in the inclination movement directions. The clearance formation section 140 includes a cone-shaped inclination surface 140a whose size of cross sectional area of the clearance gradually increases from a switch contact point 90 side to a pressure-receiving unit 136 side. FIG. 17 is a figure illustrating the pressure-receiving portion 136 of the first switch 123 maintained in the non-pressed state when the pressure-receiving portion 136 and the shaft section 137 are maintained in a neutral position without inclination. In this case, the shaft section 137 of the pressure-receiving portion 136 is maintained in a state in which the shaft section 137 can move and incline in any given directions from the neutral position.

The pressure-receiving portion 136 of the first switch 123 is a push button whose pressing surface is substantially formed in a dome shape. At the dome-shaped central position of the pressure-receiving portion 136, the first coupling section 138 with the shaft section 137 is provided. The pressure-receiving portion 136 includes an outer-peripheral-directions extension section 136a extended in an outer peripheral directions from the first coupling section 138.

The second coupling section 139 includes a flange-shaped protrusion 141 arranged to protrude from the shaft section 137 in the outer peripheral directions, and also includes a switch contact section 142 configured to be in contact with the metal dome 94 of the switch substrate 131.

When the user straightly presses the pressure-receiving portion 136 of the first switch 123 at the neutral position of FIG. 17, the switch contact section 142 presses the metal dome 94 of the switch substrate 131. Accordingly, the metal dome 94 of the first switch 123 is elastically deformed and squashed. As a result, the first switch contact point 90 of the first switch 123 and the first common contact point 92 are brought into contact, whereby the switch contact point 90 of the first switch 123 is closed. Accordingly, the transmission section of the first switch 123 of the switch substrate 131 transmits the signal of driving the apparatus.

When a user presses an end edge position of the outer-peripheral-directions extension section 136a that is displaced in any one of a given directions from the central position of the pressure-receiving portion 136, the pressure-receiving portion 136 of the first switch 123 is inclined and moved in a direction in which the pressed position resides with respect to the central position of the pressure-receiving portion 136 (pressed position direction). At this occasion, the shaft section 137 inclines and moves in one of the inclination movement directions of the pressure-receiving portion 136, and at an end edge portion on the side opposite to said one of the inclination movement directions of the pressure-receiving portion 136 with respect to the inclination movement center O of the pressure-receiving portion 136, an inclination movement support point is formed at a contact section between the protrusion 141 and the inner surface of the operation portion housing 122. When the shaft section 137 is inclined and moved about the inclination movement support point, the switch contact section 142 presses the metal dome 94 of the switch substrate 131. Accordingly, the metal dome 94 of the first switch 123 is elastically deformed and squashed. As a result, the first switch contact point 90 of the first switch 123 and the first common contact point 92 are brought into contact, whereby the switch contact point 90 of the first switch 123 is closed. Accordingly, the transmission section of the first switch 123 of the switch substrate 131 transmits the signal of driving the apparatus.

It should be noted that the second switch 124 is operated in the same manner as the first switch 123. Accordingly, the metal dome 94 of the second switch 124 is elastically deformed and squashed. As a result, the second switch contact point 91 of the second switch 124 and the second common contact point 93 are connected, whereby the switch contact point 91 of the second switch 124 is closed. Accordingly, the transmission section of the second switch 124 of the switch substrate 131 transmits the signal of driving the apparatus.

Therefore, the surgical apparatus 111 having the above configuration achieves the following effects. That is, the hand piece 114 of the surgical apparatus 111 of the present embodiment is provided with the support portion 133 configured to support the pressure-receiving portion 136 of the first switch 123 in such a manner that the pressure-receiving portion 136 of the first switch 123 can incline and move about the inclination movement center O in the operation portion housing 122. Accordingly, when the user presses a position away from the central position of the dome-shaped pressing surface of the pressure-receiving portion 136 of the first switch 123, the pressure-receiving portion 136 can be inclined and moved about the inclination movement center O in the operation portion housing 122. When the pressure-receiving portion 136 is inclined and moved with respect to the operation portion housing 122, the pressing surface of the pressure-receiving portion 136 of the first switch 123 is moved, so that the operator can press the pressing surface of the pressure-receiving portion 136 while the operator relaxes the fingers to easily press the pressure-receiving portion 136. In other words, the pressure-receiving portion 136 can be inclined and moved within the movable range of the finger of the user. This improves the ease of operation and the feeling of pressing operation when the operator operates the first switch 123.

Further, in the present embodiment, the pressure-receiving portion 136 of each of the first switch 123 and the second switch 124 is a push button whose pressing surface is formed in a dome shape. In this configuration, no matter at which position of the dome-shaped pressing surface (push button) the pressure-receiving portion 136 of each of the first switch 123 and the second switch 124 is pressed, the user can perform the switch operation adapted to cause the transmission section of the first switch 123 or the second switch 124 of the switch substrate 131 to transmit the signal of driving the apparatus. For this reason, even when the user pushes a position of the pressure-receiving portion 136 away from the central position of the pressure-receiving portion 136 during operation of the first switch 123 or the second switch 124, the switch can be operated. Therefore, the user's ease of operation can be improved, and the switch can be operated stably.

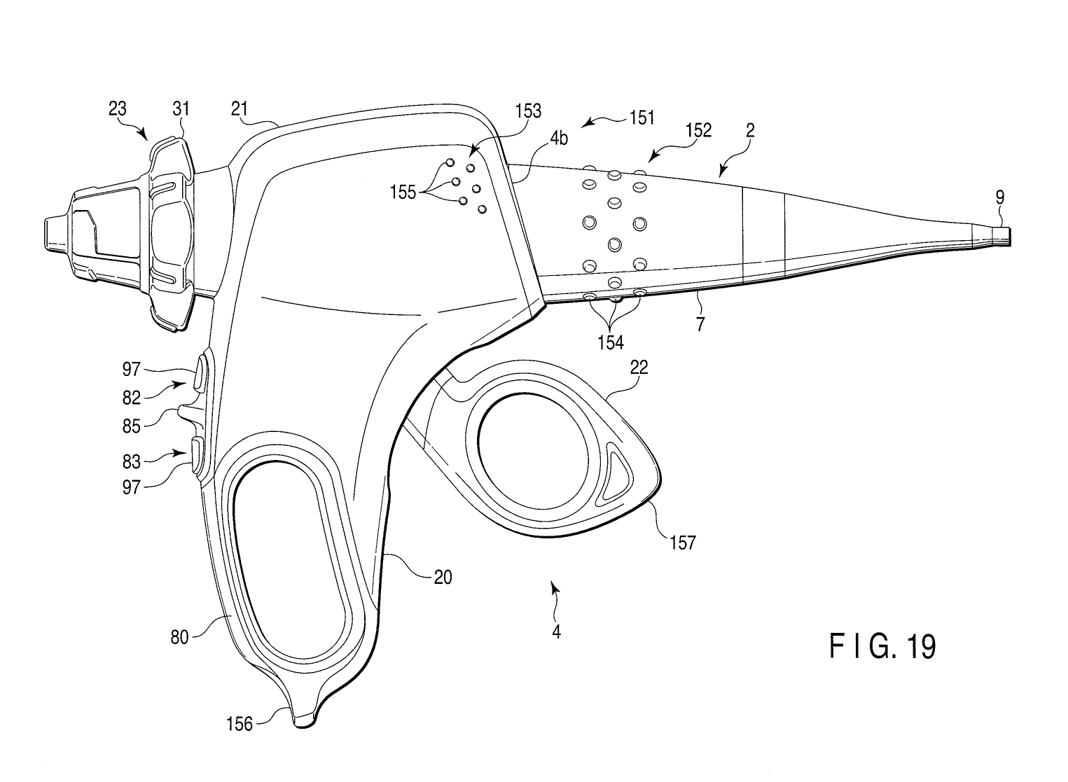

FIG. 19 is a figure illustrating a hand piece 151 of a surgical operating apparatus according to the third embodiment of the present invention. The hand piece 151 of the surgical operating apparatus according to the present embodiment is the same ultrasonic coagulation-incision treatment apparatus as that of the first embodiment (see FIGS. 1 to 15). The configurations of the basic parts are the same as the first embodiment. However, details of the configuration differ from the first embodiment. In FIG. 19, the same portions as those of the first embodiment are denoted with the same reference numerals and description thereof will not be made.

More specifically, in the present embodiment, external display portions 152, 153 having the same kind of design are provided at a coupling portion between a vibrator unit 2 and a handle unit 4. In this case, the first external display portion 152 of the vibrator unit 2 is provided on an outer peripheral surface at a distal end portion of a vibrator cover 7. In the first external display portion 152, a plurality of dot-shaped protrusions 154 are arranged along a circumferential direction.

The second external display portion 153 of the handle unit 4 is arranged at a position nearby a vibrator connection portion 4b of a holding tube 21. The second external display portion 153 is provided on both side surfaces of a proximal end portion of the holding tube 21. In the second external display portion 153, a plurality of dot-shaped protrusions 155 are arranged side by side. The first external display portion 152 of the vibrator unit 2 and the second external display portion 153 of the handle unit 4 have the same design to give the sense of unity. Accordingly, it is easy to confirm that the vibrator unit 2 and the handle unit 4 are of the same model.

In a fixed handle 20, a nail-shaped finger placing portion 156 is provided at a lower end portion of a plural-fingers insertion ring section 80 in a protruding manner. Likewise, at one end portion of a movable handle 22, a protruding portion 157 substantially in a triangular shape is provided in a protruding manner.

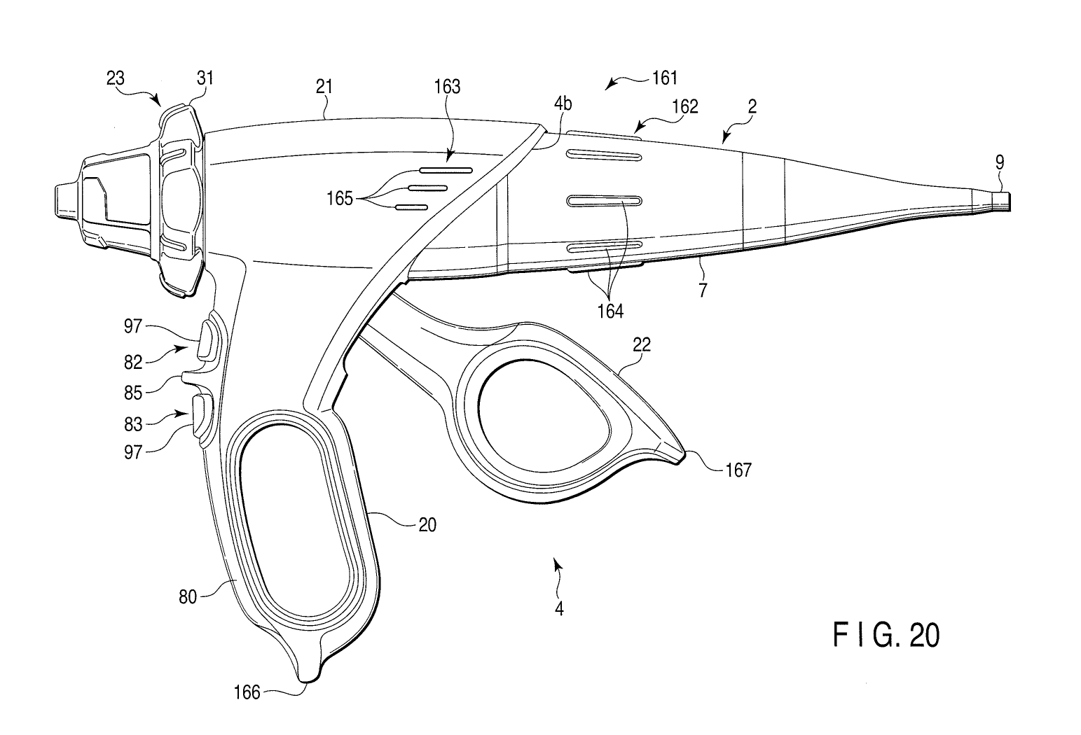

FIG. 20 is a figure illustrating a hand piece 161 of a surgical operating apparatus according to the fourth embodiment of the present invention. The hand piece 161 of the surgical operating apparatus according to the present embodiment is the same ultrasonic coagulation-incision treatment apparatus as that of the first embodiment (see FIGS. 1 to 15). The configurations of the basic parts are the same as the first embodiment. However, details of the configuration differ from the first embodiment. In FIG. 20, the same portions as those of the first embodiment are denoted with the same reference numerals and description thereof will not be made.

More specifically, in the present embodiment, external display portions 162, 163 having the same kind of design are provided at a coupling portion between a vibrator unit 2 and a handle unit 4. The external display portions 162, 163 are formed in different shapes from the external display portions 152, 153 of the hand piece 151 according to the third embodiment (see FIG. 19).

In this case, the first external display portion 162 of the vibrator unit 2 is provided on an outer peripheral surface at a distal end portion of a vibrator cover 7. In the first external display portion 162, a plurality of straight protrusions 164 are arranged along a circumferential direction side by side. Each protrusion 164 is extended substantially in parallel with an axial direction.

The second external display portion 163 of the handle unit 4 is arranged at a position nearby a vibrator connection portion 4b of a holding tube 21. The second external display portion 163 is provided on both side surfaces of a proximal end portion of the holding tube 21. In the second external display portion 163, a plurality of straight protrusions 165 are arranged side by side. The first external display portion 162 of the vibrator unit 2 and the second external display unit 163 of the handle unit 4 have the same design to give the sense of unity. Accordingly, it is easy to confirm that the vibrator unit 2 and the handle unit 4 are of the same model

In a fixed handle 20, a nail-shaped finger placing portion 166 is provided at a lower end portion of a plural-fingers insertion ring section 80 in a protruding manner. Likewise, at one end portion of a movable handle 22, a nail-shaped finger placing portion 167 is provided in a protruding manner.

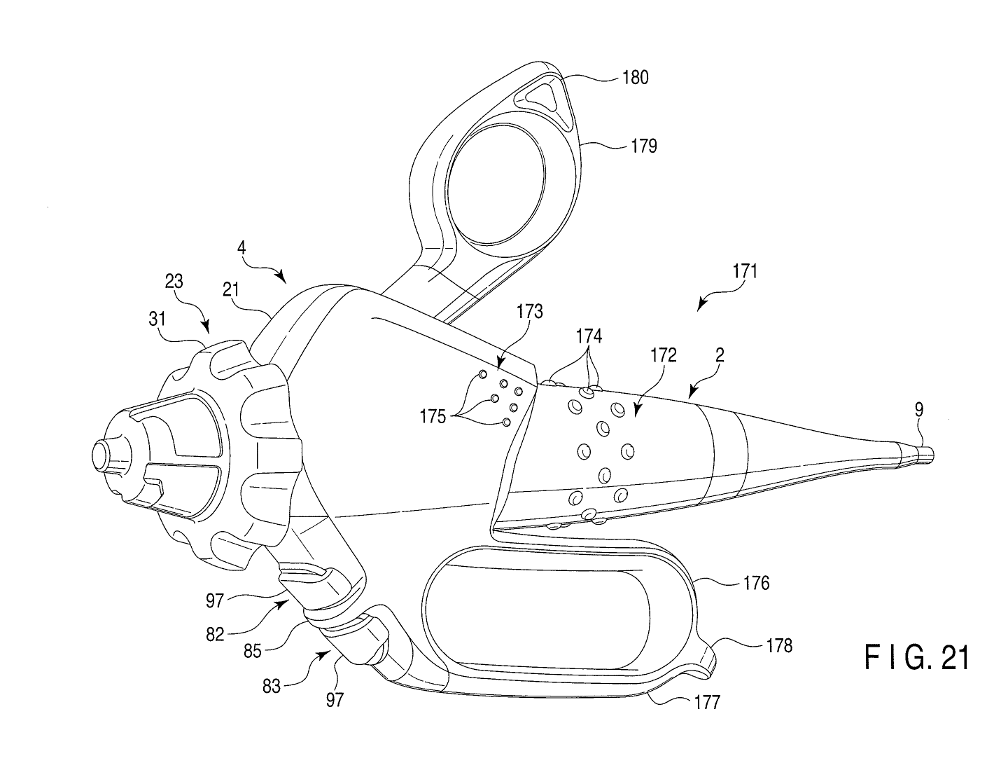

FIG. 21 illustrates a hand piece 171 of a surgical operating apparatus according to the fifth embodiment of the present invention. The present embodiment is a modification of the hand piece 151 according to the third embodiment (see FIG. 19). The hand piece 171 of the surgical operating apparatus according to the present embodiment is the same ultrasonic coagulation-incision treatment apparatus as that of the first embodiment (see FIGS. 1 to 15). The configurations of the basic parts are the same as the first embodiment. However, details of the configuration differ from the first embodiment. In FIG. 21, the same portions as those of the first embodiment are denoted with the same reference numerals and description thereof will not be made.

More specifically, in the present embodiment, external display portions 172, 173 having the same kind of design are provided at a coupling portion between a vibrator unit 2 and a handle unit 4. In this case, the first external display portion 172 of the vibrator unit 2 has substantially the same configuration as the first external display unit 152 of the vibrator unit 2 of the hand piece 151 according to the third embodiment (see FIG. 19). The first external display portion 172 of the vibrator unit 2 is provided on an outer peripheral surface at a distal end portion of a vibrator cover 7. In the first external display portion 172, a plurality of dot-shaped protrusions 174 are arranged along a circumferential direction.

The second external display portion 173 of the handle unit 4 is arranged at a position nearby vibrator connection portion 4b of a holding tube 21. The second external display portion 173 is provided on both side surfaces of a proximal end portion of the holding tube 21. In the second external display portion 173, a plurality of dot-shaped protrusions 175 are arranged side by side. The first external display portion 172 of the vibrator unit 2 and the second external display unit 173 of the handle unit 4 have the same design to give the sense of unity. Accordingly, it is easy to confirm that the vibrator unit 2 and the handle unit 4 are of the same model. The second external display portion 173 of the handle unit 4 of the hand piece 171 of the present embodiment and the second external display portion 153 of the handle unit 4 of the hand piece 151 of the third embodiment are formed in different patterns.

In the present embodiment, the configuration of the handle unit 4 is different from that of the handle unit 4 of the third embodiment. More specifically, in the handle unit 4 according to the present embodiment, a plural-fingers insertion ring section 177 of the fixed handle 176 is located below the holding tube 21 and extended to the proximal end side. At a proximal end portion of the plural-fingers insertion ring section 177, a nail-shaped finger placing portion 178 is provided in a protruding manner.

A movable handle 179 is extended above the holding tube 21. At one end portion of a movable handle 179, a protruding portion 180 substantially in a triangular shape is provided in a protruding manner.

It should be noted that the present invention is not limited to the embodiments described above. For example, the surgical apparatus may be a high-frequency surgical apparatus. The high-frequency surgical apparatus includes an electrical connection section configured to electrically connect a hand piece and a generator, the generator being configured to generate drive energy of driving the hand piece, and a conductive probe provided in the hand piece and electrically connected to the generator. A switch configured to control a high-frequency current supplied from the generator to the probe is made of the switch having the same configuration as the first switch 82 and the second switch 83 of the surgical operating apparatus according to the first embodiment (see FIGS. 1 to 15).

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An operation device configured to control driving of an apparatus, comprising:
an operation portion housing which includes a fixed handle, and a movable handle which is pivotably supported with respect to the fixed handle and which is configured to be opened and closed with respect to the fixed handle, the operation portion housing being configured to be held by a user;
a switch contact point which is provided in the fixed handle;
a switch operation portion which includes a pressure-receiving portion which is provided on a surface of a distal end side of the fixed handle and which includes a pressing surface substantially formed in an arc shape and extended from a plane, which includes a central position of the movable handle in widthwise directions of the operation portion housing perpendicular to opening/closing directions of the movable handle, to a first side and a second side opposite to the first side, the switch operation portion being configured to operate the switch contact point between an open state and a closed state in accordance with a pressing state of the pressure-receiving portion;
a support portion configured to support the pressure-receiving portion in such a manner that the pressure-receiving portion can incline and move, with respect to the fixed handle about an inclination movement center in the fixed handle, in two directions including a first inclination movement direction that is substantially perpendicular to the opening/closing directions of the movable handle and is corresponding to a tangential line direction of the pressing surface of the pressure-receiving portion and a second inclination movement direction opposite to the first inclination movement direction, the support portion being configured to support the pressure-receiving portion in such a manner that the user can press the pressure-receiving portion in a direction where the inclination movement center of the pressure-receiving portion is arranged in a state in which the pressure-receiving portion is inclined and moved and in a neutral position state in which the pressure-receiving portion is not inclined and moved; and
a restricting section configured to restrict the inclination movement directions of the pressure-receiving portion so that the pressure-receiving portion can move in two directions including the first inclination movement direction and the second inclination movement direction.

2. The operation device according to claim 1 further comprising:
an urging member configured to maintain the switch contact point in the open state during a non-pressed operation of the pressure-receiving portion in which the switch operation portion is maintained at a position where the switch contact point is maintained in the open state by the support portion; and
a switch substrate which includes a transmission section configured to transmit a signal of driving the apparatus during a close operation of the switch contact point in which the pressure-receiving portion is pressed and the switch operation portion is maintained by the support portion in such a manner that the switch operation portion can move in a direction where the switch contact point is closed against a force given by the urging member, the switch substrate being configured to close the switch contact point when pressed by the switch operation portion.

3. The operation device according to claim 2, wherein
the switch operation portion includes:
a shaft section which is disposed between the pressure-receiving portion and the switch contact point;
a first coupling section which is disposed at an end portion on a pressure-receiving portion side of the shaft section, the first coupling section coupling with the pressure-receiving portion; and
a second coupling section which is disposed at an end portion on a switch contact point side of the shaft section, the second coupling section coupling with the fixed handle, and
the support portion includes a clearance formation section configured to form a clearance between the support portion and a first coupling section side part of the shaft section, the clearance formation section being configured to cause the pressure-receiving portion to be movable in the first inclination movement direction and the second inclination movement direction about the inclination movement center of the pressure-receiving portion.

4. The operation device according to claim 3, wherein the clearance formation section includes an inclination surface whose size of cross sectional area of the clearance gradually increases from the switch contact point side to the pressure-receiving portion side.

5. The operation device according to claim 3, wherein the second coupling section includes:
a first protrusion which protrudes in the first inclination movement direction of the switch operation portion, the first protrusion being configured to form a first inclination movement support point at a contact section with an inner surface of the fixed handle when the pressure-receiving portion of the switch operation portion is inclined and moved in the second inclination movement direction;
a second protrusion which protrudes in the second inclination movement direction of the switch operation portion, the second protrusion being configured to form a second inclination movement support point at a contact section with the inner surface of the fixed handle when the pressure-receiving portion is inclined and moved in the first inclination movement direction; and
a switch contact section configured to be in contact with the switch substrate, the switch contact section being configured to press the switch substrate in the direction where the switch contact point is closed in accordance with a movement in which the shaft section of the switch operation portion inclines and moves about the first inclination movement support point in the second inclination movement direction or in accordance with a movement in which the shaft section inclines and moves about the second inclination movement support point in the first inclination movement direction.

6. The operation device according to claim 1, wherein the switch operation portion is configured to incline and move from the neutral position in a pressed position direction that is one of the first inclination movement direction and the second inclination movement direction in which a position pressed by the user is located with respect to a central position of the pressure-receiving portion.

7. The operation device according to claim 1, wherein the pressure-receiving portion of the switch operation portion can incline and move within a movable range of a finger of the user.

8. The operation device according to claim 1, wherein the switch operation portion includes a first switch operation portion and a second switch operation portion, and
the fixed handle includes a protuberance section which is provided on the surface opposite to the movable handle in a state in which the protuberance section is arranged between the pressure-receiving portion of the first switch operation portion and the pressure-receiving portion of the second switch operation portion, and on which the user can place a finger during operation.

9. The operation device according to claim 8, wherein the protuberance section includes a first side surface provided on a first switch operation portion side and a second side surface provided on a second switch operation portion side,
the first inclination movement direction and the second inclination movement direction of the first switch operation portion are in parallel with the first side surface, and
the first inclination movement direction and the second inclination movement direction of the second switch operation portion are in parallel with the second side surface.

10. A surgical apparatus comprising a hand piece which includes an operation portion configured to control driving,
wherein the operation portion includes:
an operation portion housing which includes a fixed handle, and a movable handle which is pivotably supported with respect to the fixed handle and which is configured to be opened and closed with respect to the fixed handle, the operation portion housing being configured to be held by a user;
a switch contact point which is provided in the fixed handle;
a switch operation portion which includes a pressure-receiving portion which is provided on a surface of a distal end side of the fixed handle and which includes a pressing surface substantially formed in an arc shape and extended from a plane, which includes a central position of the movable handle in widthwise directions of the operation portion housing perpendicular to opening/closing directions of the movable handle, to a first side and a second side opposite to the first side, the switch operation portion being configured to operate the switch contact point between an open state and a closed state in accordance with a pressing state of the pressure-receiving portion;
a support portion configured to support the pressure-receiving portion in such a manner that the pressure-receiving portion can incline and move, about an inclination movement center in the fixed handle with respect to the fixed handle, in two directions including a first inclination movement direction that is substantially perpendicular to the opening/closing directions of the movable handle and is corresponding to a tangential line direction of the pressing surface of the pressure-receiving portion and a second inclination movement direction opposite to the first inclination movement direction, the support portion being configured to support the pressure-receiving portion in such a manner that the user can press the pressure-receiving portion in a direction where the inclination movement center of the pressure-receiving portion is arranged in a state in which the pressure-receiving portion is inclined and moved and in a neutral position state in which the pressure-receiving portion is not inclined and moved; and
a restricting section configured to restrict the inclination movement directions of the pressure-receiving portion so that the pressure-receiving portion can move in two directions including the first inclination movement direction and the second inclination movement direction.

11. The surgical apparatus according to claim 10, wherein the operation portion:
an urging member configured to maintain the switch contact point in the open state during a non-pressed operation of the pressure-receiving portion in which the switch operation portion is maintained at a position where the switch contact point is maintained in the open state by the support portion; and
a switch substrate which includes a transmission section configured to transmit a signal of driving the apparatus during a close operation of the switch contact point in which the pressure-receiving portion is pressed and the switch operation portion is maintained by the support portion in such a manner that the switch operation portion can move in a direction where the switch contact point is closed against a force given by the urging member, the switch substrate being configured to close the switch contact point when pressed by the switch operation portion.

12. The surgical apparatus according to claim 10 which is an ultrasonic surgical apparatus further comprising:
an electrical connection section configured to electrically connect the hand piece and a generator configured to generate drive energy of driving the hand piece;
an ultrasonic vibrator which is provided in the hand piece and is electrically connected to the generator; and
an ultrasonic probe which is connected to the ultrasonic vibrator, ultrasonic vibration from the ultrasonic vibrator is configured to be transmitted to the ultrasonic probe.

13. The surgical apparatus according to claim 12, further comprising:
a sheath which covers the ultrasonic probe; and
a jaw which is attached to a distal end portion of the sheath in such a manner that the jaw can pivot with respect to a distal end portion of the ultrasonic probe.

14. The surgical apparatus according to claim 10, further comprising:
an electrical connection section configured to electrically connect the hand piece and a generator configured to generate drive energy of driving the hand piece; and
a conductive probe provided in the hand piece and electrically connected to the generator,
wherein the surgical apparatus is a high-frequency surgical apparatus capable of controlling, with the operation portion, a high-frequency current supplied from the generator to the probe.

15. The surgical apparatus according to claim 10, further comprising:
an electrical connection section configured to electrically connect the hand piece and a generator configured to generate drive energy of driving the hand piece;
an ultrasonic vibrator which is provided in the hand piece and is electrically connected to the generator; and
a conductive probe which is connected to the ultrasonic vibrator and electrically connected to the generator,
wherein the surgical apparatus is a surgical operating apparatus capable of controlling, with the operation portion, a high-frequency current supplied from the generator to the probe and an ultrasonic drive current supplied from the generator to the ultrasonic vibrator.
